(19) United States
(12) Patent Application Publication
(10) Pub. No.: US 2022/0238212 A1
(43) Pub. Date: Jul. 28, 2022

(54) METHOD AND SYSTEM FOR MANAGING MEDICAL PROCESSES

(71) Applicant: Saluspath Limited, Cambridge (GB)

(72) Inventor: Luca BIFFANI, Roma (RM) (IT)

(21) Appl. No.: 17/618,683

(22) PCT Filed: Jun. 12, 2020
(86) PCT No.: PCT/IB2020/055521

§ 371 Date: Dec. 13, 2021

(30) Foreign Application Priority Data
Jun. 13, 2019 (IT) 102019000008814

(51) Int. Cl. G16H 40/20; G16H 15/00; H04W 12/06; H04W 12/47

(52) U.S. Cl. CPC G16H 40/20; H04W 12/47; H04W 12/06; G16H 15/00

(57) ABSTRACT

Disclosed is a method for managing medical processes in a medical institution including the steps of association of the patient's identity with a portable electronic identification device with Bluetooth Low Energy technology, detection of the portable electronic identification device within the medical institution by fixed electronic detection devices with Bluetooth Low Energy technology, wherein each of the fixed electronic detection devices defines a unique detection position in the medical institution; wherein the step of detection of the portable electronic identification device includes the steps of identification of the path and timing relating to the patient's path within the medical institution; and wherein the method for managing medical processes further includes the steps of determining the actual steps of the medical process based on the steps for identifying the path and identifying the timing and comparing the pre-established and actual steps of the medical process.

### FIELD OF THE INVENTION

The present invention relates to the management of medical processes within medical institutions and, in particular, to the management and identification of the steps and timing of medical processes connected with the patient. The present invention therefore relates to the method and system for managing the aforementioned medical processes in a medical institution in such a way as to identify the actual steps of the medical process and allow the comparison thereof with the pre-established steps of the same medical process.

### PRIOR ART

It is known that the cost associated with the failure to provide a scheduled medical service, for example due to the absence of a patient with respect to the date and time of the medical service or to the lack of presence/punctuality of the medical personnel has a very important economic and social impact. In fact, economic losses for the healthcare industry are estimated at 150 billion dollars and around 125 thousand deaths in the United States alone. To allow better management of medical processes within medical institutions, ad hoc software has been developed to guarantee the integration of databases, reservations and document management of all steps of the processes associated with a medical service. Such software, however, is not able to verify adherence to the pre-established process for the patient just as it is not able to determine the characteristics of the process relating to the patient's path and timing within the medical institution.

It would therefore be desirable to have a method and a system for managing medical processes capable of minimizing the above drawbacks. In particular, it would be desirable to have a method and a system for managing medical processes capable of guaranteeing optimal management of the resources available to the medical institution and, at the same time, the possibility of carrying out a verification of the services provided to a patient as close as possible to reality and without additional management costs for the medical institution itself.

### SUMMARY OF THE INVENTION

The object of the present invention is to provide a method for managing medical processes within a medical institution capable of minimizing the aforementioned drawbacks.

In particular, the object of the present invention is to provide a method for managing medical processes within a medical institution capable of implementing automatic recognition of the steps of a medical process.

The above objects are achieved by a method for managing medical processes within a medical institution according to the appended claims.

The method for managing the medical processes within a medical institution comprises the steps of:
authentication of the identity of a patient;
verification of the medical service associated with the patient;
determination of the pre-established steps of the medical process on the basis of the medical service;

wherein the method for managing the medical processes is characterized in at it comprises the steps of:
association of the identity of the patient with a portable electronic identification device provided with Bluetooth Low Energy technology;
detection of the portable electronic identification device within the medical institution by means of a plurality of fixed electronic detection devices provided with Bluetooth Low Energy technology, wherein each of the fixed electronic detection devices defines a specific detection position in the medical institution;

wherein the step of detecting the portable electronic identification device comprises the steps of:
identification of the path of the patient within the medical institution;
identification of the timing relative to the identified path;

wherein the method for managing the medical processes further comprises the steps of:
determination of the actual steps of the medical process on the basis of the steps of identifying the path and of identifying the timing;
comparison between the pre-established steps and the actual steps of the medical process.

In this way, by means of Bluetooth Low Energy technology and the association of the patient's identity with an electronic portable identification device, the patient's movements within the medical institution can be tracked automatically and, consequently, it is always possible to automatically extract the steps of the process carried out by the patient himself. Preferably, the step of associating the identity of the patient with a portable electronic identification device is attained by associating the medical document of the patient with a beacon device suitable to transmit information by means of Bluetooth Low Energy technology, and

the step of detecting the portable electronic identification device within the medical institution by means of a plurality of fixed electronic detection devices is attained by means of a plurality of gateways suitable to receive information by means of Bluetooth Low Energy technology from the beacon device.

The acquisition of the data transmitted by the beacon using Bluetooth Low Energy technology allows tracing patient's steps and timings reducing the computational cost associated with the acquisition of the signal by the fixed electronic detection devices.

Preferably, the step of associating the patient's identity with an electronic portable identification device comprises the following steps:
reading of the medical document of the patient;
determination of the reading date of the medical document,
identification of the reserved medical service associated with the patient or of the selected medical service to be associated with the patient and of the delivery date of said reserved medical service or of said selected medical service;
association of the identity of the patient with the beacon device if at least one reserved medical service associated with the patient is present or at least one selected medical service to be associated with said patient is present and if the delivery date corresponds with the reading date of the medical document;
issuance of the beacon device.

Alternatively, the step of associating the patient's identity with an electronic portable identification device comprises the following steps:
reading of the medical document of the patient;
determination of the reading date of the medical document;
association of the identity of the patient with the beacon device;
issuance of the beacon device;
identification of at least one reserved medical service associated with the patient or of at least one selected medical service to be associated with the patient and of the delivery date of the reserved medical service or of said selected medical service.

In this way, it is possible to carry out the association of the patient's identity before check-in in the medical institution, thus using the beacon device as a queuing system and subsequent patient's monitoring and path.

Even more preferably, the method for managing medical services further comprises the step of dissociating the patient's identity from the portable electronic identification device comprising the following steps:
delivery of said beacon device;
verification of the association of said identity of the patient with said beacon device;
issuance of said medical document.

In this way, it is possible to carry out the association of the patient's identity both in the traditional way and automatically by issuing a beacon connected to the medical document.

Alternatively, the step of associating the identity of the patient with a portable electronic identification device is attained by associating the identity of the patient with a smartphone with Bluetooth Low Energy technology and provided with an application suitable to receive information by means of the Bluetooth Low Energy technology, and wherein the step of detecting the portable electronic identification device within the medical institution by means of a plurality of fixed electronic detection devices is attained by means of beacon devices suitable to transmit information by means of Bluetooth Low Energy technology to the smartphone.

The use of the smartphone for the acquisition of the data transmitted by the beacons using Bluetooth Low Energy technology allows tracing patient steps and timings without the need to provide the patient with an ad hoc device, thus reducing the cost of the institution related to the supply of the beacon devices.

Preferably, the step of identifying the patient's path within the medical institution allows the acquisition of the patient's presence in one or more of the unique detection positions of the medical institution. Even more preferably, the step of identifying the timing relative to the identified path allows the acquisition of the date and time of completion of the check-in procedure of the patient, of the time spent by the patient in one or more of the specific detection positions of the medical institution and of the date and time of completion of the check-out procedure of the patient.

Preferably, the method for managing the medical processes within a medical institution further comprises the step of determining one or more predetermined paths of the patient within said medical institution as a function of the reserved medical service associated with the patient or of the selected medical service to be associated with the patient, and further comprises the step of comparing the identified path of the patient within the medical institution with one or more of the predetermined paths of the patient within the medical institution.

In this way, it is possible to verify the movement of the patient and allow him/her to be contacted in the event of discrepancies with respect to what is predetermined by the system according to the reserved or freely accessible services. The aforementioned objects are further achieved by a system for managing medical processes within a medical institution capable of defining a patient's health performance and the pre-established steps of the medical process on the basis of the medical service.

Preferably, the method for managing the medical processes within a medical institution further comprises the step of detecting the portable electronic identification device within the medical institution by means of a plurality of mobile electronic detection devices provided with Bluetooth Low Energy technology and with an application suitable to receive information by means of the Bluetooth Low Energy technology, wherein each of the mobile electronic detection devices defines a mobile detection position in the medical institution; wherein the step of detecting the portable electronic identification device within the medical institution by means of a plurality of mobile electronic detection devices is attained by means of one or more mobile electronic detection devices suitable to:
receive information by means of Bluetooth Low Energy technology from the portable electronic identification device, wherein the received information comprise the position and the timing of the portable electronic identification device with respect to the portable electronic detection device;
store the received information; and
transmit the stored information to one or more of said fixed electronic detection devices.

In this way, it is possible to identify the patient's movements within the medical institution even in any “dark areas”, where there are no fixed electronic detection devices, maximizing the areas covered by Bluetooth Low Energy (BLE) signal and minimizing acquisition, installation and management cost of fixed BLE gateway type devices which identify a unique detection position in the institution.

The system for managing the medical processes is characterized in that it comprises:
a plurality of fixed electronic detection devices attained by cans of Bluetooth Low Energy technology, wherein each of the fixed electronic detection devices defines a specific detection position in the medical institution;
one or more portable electronic identification devices attained by means of Bluetooth Low Energy technology;

wherein each of the portable electronic identification devices is suitable to be uniquely associated with the identity of a patient,

wherein the fixed electronic detection devices are suitable to identify the portable electronic identification devices within the medical institution,

wherein the system for managing the medical processes is suitable to identify the path and the timing relative to the path of the patient within the medical institution on the basis of the portable electronic identification device, and

wherein the system for managing the medical processes is suitable to determine the actual steps of the medical process on the basis of the identified path and of said identified timing and suitable to compare the pre-established steps with the actual steps of the medical process.

Preferably, the portable electronic identification devices are constituted by beacon devices suitable to transmit information by means of Bluetooth Low Energy technology, wherein the fixed electronic detection devices are constituted by gateway devices suitable to receive information by means of Bluetooth Low Energy technology from the beacon devices, and wherein the system for managing the medical processes is suitable to identify the path and the timing relative to the path of the patient within the medical institution on the basis of the information received by the gateway devices from the beacon device.

Alternatively, the portable electronic identification devices are constituted by smartphones with Bluetooth Low Energy technology and provided with an application suitable to receive information by means of the Bluetooth Low Energy technology,

wherein the fixed electronic detection devices are constituted by beacon devices suitable to transmit information by means of Bluetooth Low Energy technology to the smartphone, and

wherein the system for managing the medical processes is suitable to identify the path and the timing relative to the path of the patient within the medical institution on the basis of the information sent by the beacon devices to the smartphone.

Preferably, the system for managing the medical processes within a medical institution further comprises one or more portable electronic detection devices attained by means of Bluetooth Low Energy technology and provided with an application suitable to receive information by means of said Bluetooth Low Energy technology, wherein each of the portable electronic detection devices defines a mobile detection position in the medical institution;

wherein the portable electronic detection devices are suitable to:
receive information by means of Bluetooth Low Energy technology from the portable electronic identification device, wherein e received information comprise the position and the timing of the portable electronic identification device with respect to the portable electronic detection device;
store the received information; and
transmit the stored information to one or more of said fixed electronic detection devices.

### DESCRIPTION OF THE FIGURES

These and further features and advantages of the present invention will become apparent from the description of the preferred embodiments, illustrated by way of non-limiting example in the accompanying figures, in which:

FIG. 1 is a schematic view of the method in which the patient's identity is associated with the beacon device, according to the present invention;

FIG. 2 is a schematic view of the method in which the patient's identity is associated with the smartphone, according to the present invention.

### DETAILED DESCRIPTION OF THE INVENTION

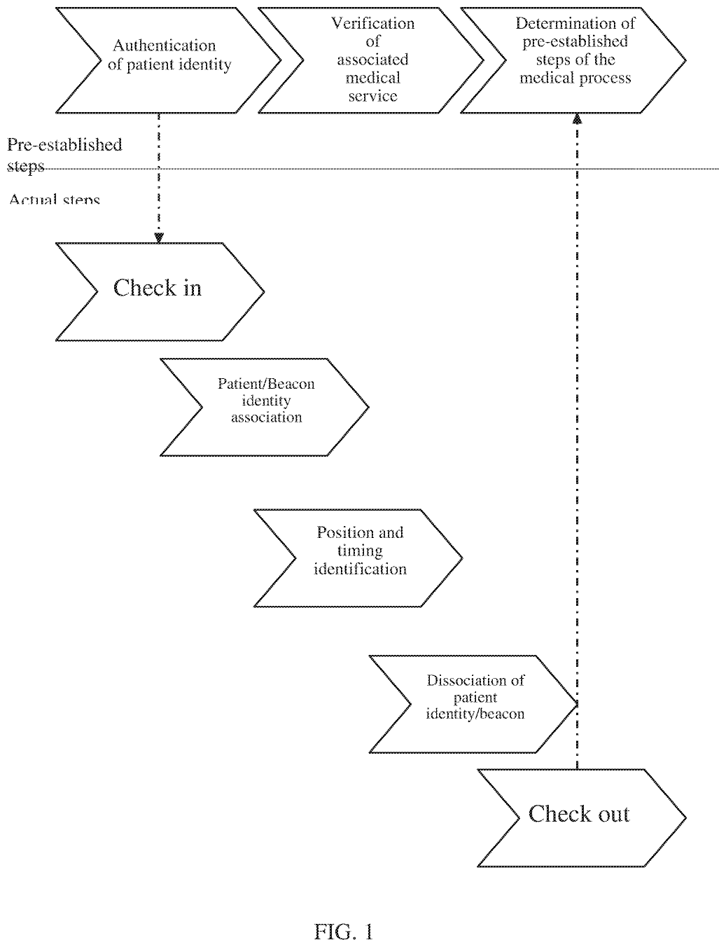

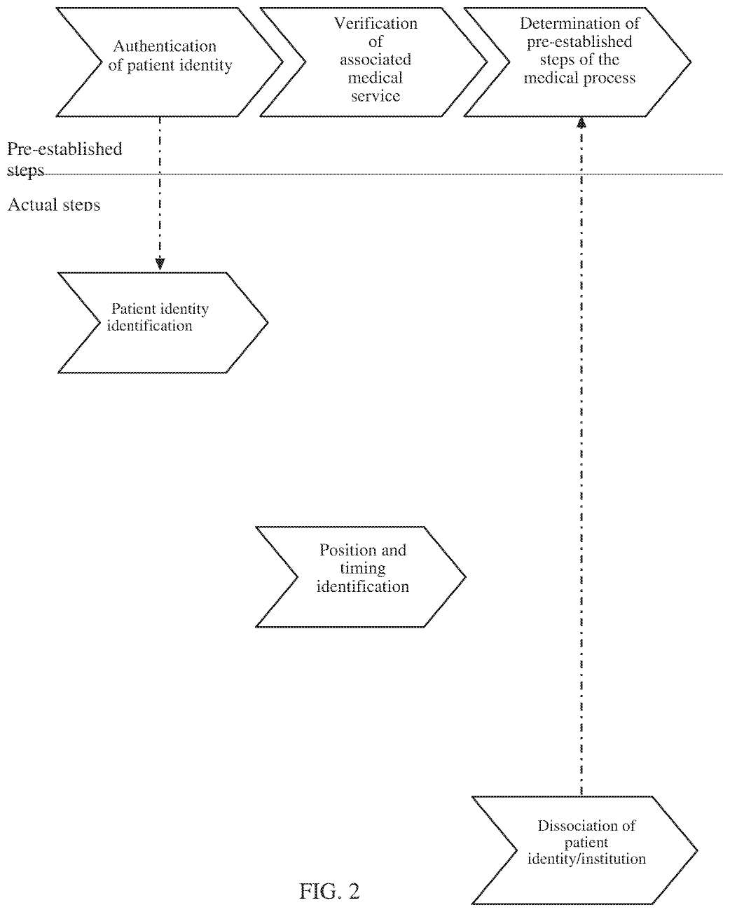

With reference to FIGS. 1 and 2, the method and system for managing medical processes within a medical institution, according to the present invention, are described below.

The term “medical process” means, in the present invention, the set of activities carried out within a medical institution which create value for the patient or for the medical institution itself by transforming the input resources of the process into a final output product of the process, typically a medical service.

The object of the present invention is to provide a method and a system for managing medical processes within a medical institution capable of implementing automatic recognition of the steps of a medical process. In particular, the system for managing medical processes within a medical institution capable of defining a patient's health performance and the pre-established steps of the medical process on the basis of the medical service.

To achieve the intended objects, the system for managing the medical processes is implemented using Bluetooth Low Energy technology, i.e. through a wireless personal area network technology with the aim of providing significantly reduced energy consumption and cost, maintaining a communication interval similar to the traditional Bluetooth technology.

The system object of the present invention comprises a plurality of fixed electronic detection devices implemented using Bluetooth Low Energy technology, in which each of the fixed electronic detection devices defines a unique detection position in the medical institution and one or more portable electronic identification devices implemented using Bluetooth Low Energy technology.

Each of the aforementioned portable electronic identification devices is capable of being uniquely associated with the identity of a patient, while the fixed electronic detection devices are suitable to detect the portable electronic identification devices within the medical institution.

The system for managing medical processes object of the present invention is, therefore, able to identify the path and timing relating to the patient's path within the medical institution on the basis of the portable electronic identification device, and to determine the actual steps of the medical process on the basis of the path and timing identified, comparing the pre-established steps with the actual steps of the medical process.

According to the present invention, the technological structure of the system may be implemented by providing a single mobile beacon or, alternatively, by a plurality of fixed beacons, as described below in relation to the two preferred embodiments illustrated in connection with the method according to the present invention in FIGS. 1 and 2.

Bluetooth-based Beacon technology allows Bluetooth devices to transmit and receive small messages within short distances. In brief, it consists of two parts: a presenter (a beacon device) and a receiver. The presenter always comes up with the same message, while the receiver detects these beacon sensors.

Standard beacon information consists of a ULM), and only a major or minor value. For example: Ulna 139407F30-F5F8-466E-AFF9-25556B57FE6D Major ID: 1 Minor ID: 2 The transmitter does nothing but send this information every split second. The UUID is a unique identifier.

The system for managing the medical processes is therefore suitable to identify the path and the timing relative to the path of the patient within the medical institution on the basis of the information received by the gateway devices from the beacon device. In particular, the method for managing medical processes within a medical institution includes the traditional steps associated with the provision of a medical service and the additional steps necessary for the identification of the aforementioned path and timing of each patient.

Traditional steps include authenticating a patient's identity, verifying the patient's medical service and determining the pre-established steps of the medical process based on the medical service.

Furthermore, the method for managing medical processes, object of the present invention, includes the steps of association of the patient's identity with an electronic portable identification device provided with Bluetooth Low Energy technology, detection of the electronic portable identification device within the medical institution by means of a plurality of fixed electronic detection devices provided with Bluetooth Low Energy technology, in which each of the fixed electronic detection devices defines a unique detection position in the medical institution. In particular, the step of detection of the portable electronic identification device comprises the steps for identifying the patient's path within the medical institution and identifying the timing relating to the identified path.

The method for managing health processes, according to the present invention, further includes the steps of determining the actual steps of the medical process on the basis of the path identification and timing identification steps and the comparison between the pre-established and the actual steps of the medical process.

In this way, by means of Bluetooth Low Energy technology and the association of the patient's identity with an electronic portable identification device, the patient's movements within the medical institution can be tracked automatically and, consequently, it is always possible to extract automatically the steps of the process carried out by the patient himself, as described in more detail below.

According to the method illustrated in FIG. 1, in the embodiment of the relative system, portable electronic identification devices consist of beacon devices adapted to transmit information using Bluetooth Low Energy technology, while fixed electronic detection devices consist of gateways devices adapted to receive information via Bluetooth Low Energy technology from beacon devices.

According to the technological structure described above, for example, each mobile beacon device defines a unique UUID for use with all the gateway devices arranged within the medical institution. In particular, within the institution, they may be configured in such a way that a different minor value is used for each gateway device. For example, the minor value 1 at the check-in station, the minor value 2 at the check-out station and increasing values at each waiting room, service station, or pedestrian crossings depending on the position within the medical institution.

The system for managing the medical processes is therefore suitable to identify the path and the timing relative to the path of the patient within the medical institution on the basis of the information received by the gateway devices from the beacon device.

According to the method shown in FIG. 1, the step of associating the identity of the patient with a portable electronic identification device is attained by associating the medical document of the patient with a beacon device suitable to transmit information by means of Bluetooth Low Energy technology, and

the step of detecting the portable electronic identification device within the medical institution by means of a plurality of fixed electronic detection devices is attained by means of a plurality of gateways suitable to receive information by means of Bluetooth Low Energy technology from the beacon device.

Alternatively, the step of association of the patient's identity with the portable electronic identification device may be carried out by means of facial recognition of the patient, as an alternative or in addition to the use of the patient's medical document.

With the information transmitted by each beacon, the system, after reading it, can tell how close (or far) the beacons are with respect to the gateways positioned and, consequently, determine all the steps of the process actually carried out by the patient, comparing them with the steps of the pre-established process for a specific medical service.

The acquisition of the data transmitted by the beacon using Bluetooth Low Energy technology allows tracing patient's steps and timings reducing the computational cost associated with the acquisition of the signal by the fixed electronic detection devices.

The step of association of the patient's identity with a portable electronic identification device is therefore carried out during a check-in step through the traditional approach with an operator or through an automatic dispenser of beacon devices. In any case, such a step of association of the patient's identity with an electronic portable identification device includes the following steps of reading the patient's medical document, determining the reading date of the medical document, identifying the reserved medical service associated with the patient or the selected medical service to be associated with the patient and the delivery date of the reserved medical service or the selected medical service, associating the patient's identity to the beacon device if there is at least one reserved medical service associated with the patient or at least one selected medical service to be associated with the patient and if the delivery date corresponds to the reading date of the medical document and, finally, issuing the beacon device.

The medical document associated with the patient is therefore retained by the operator or by the automatic dispenser from the check-in step until the end of the medical process, determined by the check-out step. In this case, the step of dissociation of the patient's identity from the portable electronic identification device includes the steps of delivery of the beacon device, verification of the association of the patient's identity with the beacon device and issuance of the medical document.

In this way, it is possible to carry out the association of the patient's identity both in the traditional way and automatically by issuing a beacon connected to the medical document.

Considering a patient who has reserved a medical service, the steps of the process according to the present invention are hypothesized below with respect to the steps of the process connected with the medical service to be provided.

Upon entering the medical institution, the patient is identified by identifying the medical services to be provided. In the case of medical services to be delivered on the same day, check-in takes place, i.e. the patient is given the beacon device which from that moment will begin to identify the patient's positioning and timing within the institution. Therefore, the step of identifying the patient's path within the medical institution allows the acquisition of the patient's presence in one or more of the unique detection positions of the medical institution. Even more preferably, the step of identifying the timing relative to the identified path allows the acquisition of the date and time of completion of the check-in procedure of the patient, of the time spent by the patient in one or more of the specific detection positions of the medical institution and of the date and time of completion of the check-out procedure of the patient.

Therefore, the patient is first detected by the gateway device located in the check-in area, identifying the first step of the actual process, in which the movement with respect to the check-in area allows you to determine the date and time of completion of the check-in procedure. During movement within the medical institution, the patient is detected by the different gateway devices located within the medical institution, by relating the positioning and timing of the path taken. For example, at a waiting room the method and the system according to the present invention allow determining arrival time and residence time in the waiting room. In the same way, at the examination room, the method and the system according to the present invention allow determining arrival time and residence time in the examination room, that is the actual working time necessary for the examination. The aforementioned detections also allow determining the absence or presence of a patient with respect to the scheduled examination, as well as the punctuality of the medical worker with respect to the scheduled examination time. At the end of the examination and during movement within the medical institution, the patient is again detected by the different gateway devices located within the medical institution, by relating the positioning and timing of the path taken. Finally, the patient is detected by the gateway device located in the check-out area, identifying the last step of the actual process, in which the movement with respect to the check-out area allows you to determine the date and time of completion of the check-out procedure itself.

According to a further aspect, it is possible to consider the hypothesis of a patient who does not have any reserved medical services and who wants to access free-access medical services. The steps of the process according to the present invention are hypothesized below with respect to the steps of the process connected with the medical service to be provided.

Upon entering the medical institution, the patient is identified by identifying the medical services selected by him/her, of the free access type. In the case of availability of free-access medical services to be delivered on the same day, check-in takes place, i.e. the patient is given the beacon device which from that moment will begin to identify the patient's positioning and timing within the institution. Therefore, the step of identifying the patient's path within the medical institution allows the acquisition of the patient's presence in one or more of the unique detection positions of the medical institution. Even more preferably, the step of identifying the timing relative to the identified path allows the acquisition of the date and time of completion of the check-in procedure of the patient, of the time spent by the patient in one or more of the specific detection positions of the medical institution and of the date and time of completion of the check-out procedure of the patient.

Therefore, all the steps described above are repeated until the patient is detected by the gateway device located in the check-out area, identifying the last step of the actual process, in which the movement with respect to the check-out area allows you to determine the date and time of completion of the check-out procedure itself.

According to a further embodiment, it is possible that the beacon device is issued to the patient before check-in, thus performing not only the association of the patient's identity but also defining a queuing system and subsequent monitoring and path of the patient.

The steps of the process according to the present invention are hypothesized below with respect to the steps of the process connected with the medical service to be provided.

Upon entering the medical institution, the patient is identified by reading the medical document and the beacon device associated with it is delivered, which from that moment will begin to identify the patient's positioning and timing within the institution.

The beacon issued will function as a queuing system in the check-in step, in the payment step and in any subsequent step of the delivery of medical services (i.e. management of queues for reserved examinations and/or freely delivered). Only subsequently will the reserved or free-access medical services be associated with the beacon already issued. The issued beacon can, therefore, be used as a queue elimination system, that is, capable of determining the order of access of patients to the reserved or free access services without any need for further interaction with the patient or for requesting external management systems for queues.

In the check-in area, the beacon therefore becomes an integral part of the process and of the system, also fully tracing the check-in or waiting for check-in step. Therefore, not only will the date and time of completion of the issuance of the beacon device be determined, but also the date and time of the patient's arrival at the check-in area and, therefore, the waiting timing.

As described above, the step of identifying the patient's path within the medical institution allows the acquisition of the patient's presence in one or more of the unique detection positions of the medical institution. Even more preferably, the step of identifying the timing relative to the identified path allows the acquisition of the date and time of completion of the check-in procedure of the patient, of the time spent by the patient in one or more of the specific detection positions of the medical institution and of the date and time of completion of the check-out procedure of the patient.

Therefore, all the steps described above are repeated until the patient is detected by the gateway device located in the check-out area, identifying the last step of the actual process, in which the movement with respect to the check-out area allows you to determine the date and time of completion of the check-out procedure itself.

According to the method illustrated in FIG. 2, in the embodiment of the relative system, portable electronic identification devices consist of smartphones with Bluetooth Low Energy technology and provided with an application adapted to receive information using Bluetooth Low Energy technology, while fixed electronic detection devices consist of beacon devices adapted to transmit information via Bluetooth Low Energy technology to the smartphone.

As described for the structure of the previous embodiment, for example, each fixed beacon device arranged within the medical institution defines a unique UUID for interacting with the smartphone associated with the patient. In particular, within the institution, they may be configured in such a way that a different minor value is used for each beacon device. For example, the minor value 1 at the check-in station, the minor value 2 at the check-out station and increasing values at each waiting room, service station, or pedestrian crossings depending on the position within the medical institution. The smartphone integrates Bluetooth Low Energy technology and the APIs that deal with its functionality are part of the operating system.

The system for managing the medical processes is therefore suitable to identify the path and the timing relative to the path of the patient within the medical institution on the basis of the information sent by the beacon devices to the smartphone.

According to the method shown in FIG. 2, the step of associating the identity of the patient with a portable electronic identification device is attained by associating the identity of the patient with a smartphone with Bluetooth Low Energy technology and provided with an application suitable to receive information by means of the Bluetooth Low Energy technology, and

The step of detecting the portable electronic identification device within the medical institution by means of a plurality of fixed electronic detection devices is attained by means of beacon devices suitable to transmit information by means of Bluetooth Low Energy technology to the smartphone.

It is also possible to use the smartphone as an active element, or as a beacon device if the medical institution is provided with gateway devices, as described in the embodiment of FIG. 1.

With the information transmitted by each beacon, received or transmitted by the smartphone, the system, can determine all the steps of the process actually carried out by the patient, comparing them with the steps of the pre-established process for a specific medical service.

The use of the smartphone for the acquisition of the data transmitted by the beacons using Bluetooth Low Energy technology allows tracing patient steps and timings without the need to provide the patient with an ad hoc device, thus reducing the cost of the institution.

As described above, the step of identifying the patient's path within the medical institution allows the acquisition of the patient's presence in one or more of the unique detection positions of the medical institution. Furthermore, the timing identification step relative to the identified path allows the acquisition of the time spent by the patient in one or more of the unique detection positions of the medical institution.

Let us consider a patient who goes to the medical institution even without a previous reservation of a medical service, the steps of the process according to the present invention are hypothesized below with respect to the steps of the process connected with the medical service to be provided.

In particular, with respect to the previous embodiment, the check-in and check-out steps may be completely eliminated from the process, independently determined with the interaction of the smartphone within the medical institution, or by integrating useful information for a queue management system.

Upon entering the medical institution, the patient is detected by identifying the smartphone associated with him/her and provided with Bluetooth Low Energy technology and from that moment the system will begin to identify the patient's positioning and timing within the institution. The step of identifying the patient's path within the medical institution allows the acquisition of the patient's presence in one or more of the unique detection positions of the medical institution. Even more preferably, the timing identification step relative to the identified path allows the acquisition of the date and time of completion of the time spent by the patient in one or more of the unique detection positions of the medical institution.

Therefore, the patient is first detected by the Bluetooth Low Energy device placed in the entrance gate of the medical institution, identifying the first step of the actual process as well as the date and time of access to the medical institution. During movement within the medical institution, the patient is detected by the different devices located within the medical institution, by relating the positioning and timing of the path taken. For example, at a waiting room the method and the system according to the present invention allow determining arrival time and residence time in the waiting room. In the same way, at the examination room, the method and the system according to the present invention allow determining arrival time and residence time in the examination room, that is the actual working time necessary for the examination. The aforementioned detections also allow determining the absence or presence of a patient with respect to the scheduled examination, as well as the punctuality of the medical worker with respect to the scheduled examination time. At the end of the examination and during movement within the medical institution, the patient is again detected by the different devices located within the medical institution, by relating the positioning and timing of the path taken. Finally, the patient is detected by the Bluetooth Low Energy device placed at the exit gate, identifying the last step of the actual process, i.e. date and time of exit from the medical institution.

The same considerations may apply, Mutatis mutandis, if a patient is considered to go to the medical institution with at least one reserved medical service to be performed on the date of access to the aforementioned institution, as already described above for the first embodiment.

In addition, the embodiments described above may include the possibility of tracking the patient within the medical institution so as to maximize the areas covered by Bluetooth Low Energy (BLE) signal but, at the same time, minimizing acquisition, installation and management costs of fixed electronic detection devices, for example BLE gateways that identify a unique detection position in the medical institution.

To this end, the system for managing the medical processes within a medical institution further comprises one or more portable electronic detection devices attained by means of Bluetooth Low Energy technology and provided with an application suitable to receive information by means of said Bluetooth Low Energy technology, wherein each of the portable electronic detection devices defines a mobile detection position in the medical institution;

The aforementioned portable electronic detection devices therefore constitute a plurality of mobile gateways provided with BLE technology. These portable electronic detection devices may be implemented, for example, by means of a smartphone provided with a proprietary app, capable of receiving “beacon” signals, i.e. information which includes the position and timing of the portable electronic identification device detected within the medical institution compared to the portable electronic detection device. Furthermore, the aforesaid portable electronic detection devices allow storing the information received and transmitting the same information to the fixed electronic detection devices.

Portable electronic detection devices may therefore be assigned to part of the internal staff, typically on the move in the medical institution and which, therefore, allow defining mobile detection positions within the medical institution itself. The position of the staff provided with the portable electronic detection device is always identified, on a probabilistic basis, with respect to the path between two points necessarily covered by fixed electronic detection devices.

According to an embodiment, the electronic fixed detection devices may be positioned exclusively in the main areas of interest for the identification of the patient within the medical institution, for example check-in, waiting, visit and check-out areas. In areas without fixed electronic detection devices, patient detection is carried out by means of type mobile gateways and/or by smartphones provided with BLE technology that define the electronic detection mobile devices as described above.

Preferably, the method for managing the medical processes within a medical institution further comprises the step of detection of the electronic portable identification device within the medical institution by means of one or more of the mobile electronic detection devices, as described below.

The patient provided with the mobile electronic identification device is detected by the fixed electronic detection device positioned, for example, in the check-in area, as described in the previous embodiments.

During the movement in the medical institution, the patient could therefore travel through areas not provided with fixed electronic detection devices. During this movement, the patient could meet other subjects, for example staff of the medical institution, provided with the aforementioned mobile electronic detection devices. During the meeting, the mobile electronic detection device receives information via Bluetooth Low Energy technology from the mobile electronic identification device, or from the beacon device associated with the patient and/or from the smartphone associated with the patient. In particular, the information identifies the mobile electronic identification device and the timing of reception of the aforementioned information.

The information received is therefore stored on the mobile electronic detection device until the latter reaches the area of a fixed electronic detection device.

The mobile electronic detection device therefore transmits the information stored to the relative fixed electronic detection device, for example the fixed gateway, allowing the collection of the data identified for the patient in areas not covered by fixed electronic detection devices.

The system and method for managing medical processes within a medical institution therefore allow, on a probabilistic basis, identifying the path taken by the employee and, also according to the timing of the meeting with the patient, the meeting place. Consequently, the system and the method for managing medical processes within a medical institution allow, therefore, always on a probabilistic basis, identifying the path taken by the patient according to the relative position and timing of meeting with the employee.

The detection with respect to the fixed electronic detection devices is therefore defined as in the previous embodiments, both at the other fixed gateways and in the check-out area.

According to a further embodiment, it is possible that the mobile electronic detection devices may communicate with each other to allow the transmission of the information collected by the mobile electronic identification devices, thus defining a bridge system until the information is forwarded to the first fixed electronic detection device. This transmission of information allows the system and the method, again on a probabilistic basis, to identify the path taken by the employee between two fixed electronic detection devices and, also according to the timing of the meeting with the patient, the meeting place, considering the data with respect to all the electronic detection devices.

In this way, it is possible to identify the patient's movements within the medical institution even in any “dark areas”, where there are no fixed electronic detection devices.

The methods and systems described above for the management of medical processes within a medical institution may also find useful integration with methods and systems for managing and verifying the compliance of medical services.

In this regard, the aforementioned systems and methods for managing and verifying compliance of medical services are described below, integrated with the systems and methods for managing the medical processes described above.

For all the aforementioned embodiments, it is possible to define a step of determining one or more predetermined paths of the patient within said medical institution as a function of the reserved medical service associated with the patient or of the selected medical service to be associated with the patient. The predetermined paths may, therefore, be stored on suitable storage media of the system, in such a way as to allow their subsequent possibility of reuse for possible comparisons.

Subsequently, it is possible define the step of comparing the identified path of the patient within the medical institution with one or more of the above predetermined paths of the patient within the medical institution.

In this way, it is possible to verify the movement of the patient and allow him/her to be contacted in the event of discrepancies with respect to what is predetermined by the system according to the reserved or freely accessible services. In this regard, the information regarding the patient's contact possibilities (for example, telephone number, etc.) may also be stored on appropriate storage media of the system or collected during the patient's access to the medical institution (at each access or the first access).

In this way, it is possible to trace back within the pre-established steps of the medical process (presence, path and timing of the patient), the actual ones that deviate from it, for example as a consequence of the patient's delayed arrival in the medical institution and identification of a path that differs from what is predetermined. Using the contact information, it is possible to communicate to the patient information useful for correcting the path, for example through information transmitted on a mobile device (fir example through a special APP), or by calling the patient himself/herself.

According to a further aspect, the system allows the verification of late patients and also the calculation of the time of acceptance and delay with respect to the examination time, to free a slot and make the availability within the medical institution more efficient. For example, it is possible to determine a free slot if the average examination time is less than the average acceptance time to which to add the average travel time. Furthermore, as a consequence of the non-presence/arrival of the patient who reserved the medical service, as foreseen by the work plan of the medical institution and according to the pre-established timing, it is possible to communicate (for example through an application) the availability of free slots for medical services to patients, as well as to the medical professionals concerned.

Furthermore, if the system and the method according to the present invention are used in connection with a proprietary database of the medical institution, either local or remote, it is possible to allow an operational connection such as to obtain an exchange of information at the check-in step and, possibly, at each step deemed relevant for the acquisition of information. For example, it is possible to automatically allow management of information relating to the presence/absence of the patient at each medical service to be provided, ensuring the integrity of use of the available slot and the two-way relationship between the service p d and the payment to be made. In this way, the system and method according to the present invention may define a data structure of the “blockchain” type, in which it is possible to define a chronological linking of the steps and in which integrity is guaranteed by the automatic management underlying the present invention.

1. Method for managing the medical processes within a medical institution comprising the steps of:
authentication of the identity of a patient;
verification of the medical service associated with said patient;
determination of the pre-established steps of the medical process on the basis of said medical service;
wherein said method for managing the medical processes comprises:
associating said identity of said patient with a portable electronic identification device provided with Bluetooth Low Energy technology;
detecting said portable electronic identification device within said medical institution by means of a plurality of fixed electronic detection devices provided with Bluetooth Low Energy technology, wherein each of said fixed electronic detection devices defines a specific detection position in said medical institution;
wherein said step of detecting said portable electronic identification device comprises the steps of:
identifying the path of said patient within said medical institution;
identifying the timing relative to said identified path;
wherein said method for managing the medical processes further comprises the steps of:
determining the actual steps of the medical process on the basis of said steps of identifying the path and of identifying the timing;
comparing said pre-established steps and said actual steps of the medical process.

2. The method for managing the medical processes within a medical institution according to claim 1, wherein said step of associating said identity of said patient with a portable electronic identification device is attained by associating the medical document of said patient with a beacon device suitable to transmit information by means of Bluetooth Low Energy technology, and
wherein said step of detecting said portable electronic identification device within said medical institution by means of a plurality of fixed electronic detection devices is attained by means of a plurality of gateways suitable to receive information by means of Bluetooth Low Energy technology from said beacon device.

3. The method for managing the medical processes within a medical institution according to claim 2, wherein said step of associating said identity of said patient with a portable electronic identification device comprises the following steps:
reading of the medical document of said patient;
determining the reading date of the medical document;
identifying the reserved medical service associated with said patient or of the selected medical service to be associated with said patient and of the delivery date of said reserved medical service or of said selected medical service;
associating said identity of the patient with said beacon device if at least one reserved medical service associated with said patient is present or at least one selected medical service to be associated with said patient is present and if said delivery date corresponds with said reading date of the medical document;
issuing said beacon device.

4. The method for managing the medical processes within a medical institution according to claim 2, wherein said step of associating said identity of said patient with a portable electronic identification device comprises the following steps:
reading of the medical document of said patient;
determining the reading date of the medical document;
associating said identity of the patient with said beacon device;
issuing said beacon device;
identifying at least one reserved medical service associated with said patient or of at least one selected medical service to be associated with said patient and of the delivery date of said reserved medical service or of said selected medical service.

5. The method for managing the medical processes within a medical institution according to claim 3, further comprising the step of dissociating said identity of said patient from the portable electronic identification device comprising the following steps:
delivering said beacon device;
verifying the association of said identity of the patient with said beacon device;
issuing said medical document.

6. The method for managing the medical processes within a medical institution according to claim 1, wherein said step of associating said identity of said patient with a portable electronic identification device is attained by associating the identity of said patient with a smartphone with Bluetooth Low Energy technology and provided with an application suitable to receive information by means of said Bluetooth Low Energy technology, and
wherein said step of detecting said portable electronic identification device within said medical institution by means of a plurality of fixed electronic detection devices is attained by means of beacon devices suitable to transmit information by means of Bluetooth Low Energy technology to said smartphone.

7. The method for managing the medical processes within a medical institution according to claim 1, wherein said step of identifying the path of said patient within said medical institution allows the acquisition of the presence of said patient in one or more of said specific detection positions of said medical institution.

8. The method for managing the medical processes within a medical institution according to claim 1, wherein said step of identifying the timing relative to said identified path allows the acquisition of the date and time of completion of the check-in procedure of said patient, of the time spent by said patient in one or more of said specific detection positions of said medical institution and of the date and time of completion of the check-out procedure of said patient.

9. The method for managing the medical processes within a medical institution according to claim 1, further comprising the step of determining one or more predetermined paths of said patient within said medical institution as a function of said reserved medical service associated with said patient or of said selected medical service to be associated with said patient, and
further comprising the step of comparing said identified path of said patient within said medical institution with one or more of said predetermined paths of said patient within said medical institution.

10. The method for managing the medical processes within a medical institution according to claim 1, further comprising the step of detecting said portable electronic identification device within said medical institution by means of a plurality of mobile electronic detection devices provided with Bluetooth Low Energy technology and with an application suitable to receive information by means of said Bluetooth Low Energy technology, wherein each of said mobile electronic detection devices defines a mobile detection position in said medical institution;
wherein said step of detecting said portable electronic identification device within said medical institution by means of a plurality of mobile electronic detection devices is attained by means of one or more mobile electronic detection devices suitable to:
receive information by means of Bluetooth Low Energy technology from said portable electronic identification device, wherein the received information comprise the position and the timing of said portable electronic identification device with respect to said portable electronic detection device;
store said received information; and
transmit said stored information to one or more of said fixed electronic detection devices.

11. System for managing the medical processes within a medical institution suitable to define the medical service of a patient and the pre-established steps of the medical process on the basis of said medical service, wherein said system for managing the medical processes comprises:
a plurality of fixed electronic detection devices attained by means of Bluetooth Low Energy technology, wherein each of said fixed electronic detection devices defines a specific detection position in said medical institution;
one or more portable electronic identification devices attained by means of Bluetooth Low Energy technology;
wherein each of said portable electronic identification devices is suitable to be uniquely associated with the identity of a patient,
wherein said fixed electronic detection devices are suitable to detect said portable electronic identification devices within said medical institution,
wherein said system for managing the medical processes is suitable to identify the path and the timing relative to said path of said patient within said medical institution on the basis of said portable electronic identification device, and
wherein said system for managing the medical processes is suitable to determine the actual steps of the medical process on the basis of said identified path and of said identified timing and suitable to compare said pre-established steps with said actual steps of the medical process.

12. System for managing the medical processes within a medical institution according to claim 11, wherein said portable electronic identification devices are constituted by beacon devices suitable to transmit information by means of Bluetooth Low Energy technology,
wherein said fixed electronic detection devices are constituted by gateway devices suitable to receive information by means of Bluetooth Low Energy technology from said beacon devices, and
wherein said system for managing the medical processes is suitable to identify the path and the timing relative to said path of said patient within said medical institution on the basis of said information received by said gateway devices from said beacon device.

13. System for managing the medical processes within a medical institution according to claim 11, wherein said portable electronic identification devices are constituted by smartphones with Bluetooth Low Energy technology and provided with an application suitable to receive information by means of said Bluetooth Low Energy technology,
wherein said fixed electronic identification devices are constituted by beacon devices suitable to transmit information by means of Bluetooth Low Energy technology to said smartphone, and
wherein said system for managing the medical processes is suitable to identify the path and the timing relative to said path of said patient within said medical institution on the basis of said information sent by said beacon devices to said smartphone.

14. System for managing the medical processes within a medical institution according to claim 11, further comprising one or more portable electronic detection devices attained by means of Bluetooth Low Energy technology and provided with an application suitable to receive information by means of said Bluetooth Low Energy technology, wherein each of said portable electronic detection devices defines a mobile detection position in said medical institution;
wherein said portable electronic detection devices are suitable to:
receive information by means of Bluetooth Low Energy technology from said portable electronic identification device, wherein the received information comprise the position and the timing of said portable electronic identification device with respect to said portable electronic detection device;
store said received information; and
transmit said stored information to one or more of said fixed electronic detection devices.

15. The method for managing the medical processes within a medical institution according to claim 4, further comprising the step of dissociating said identity of said patient from the portable electronic identification device comprising the following steps:
delivering said beacon device;
verifying the association of said identity of the patient with said beacon device;
issuing said medical document.

16. The method for managing the medical processes within a medical institution according to claim 2, wherein said step of identifying the path of said patient within said medical institution allows the acquisition of the presence of said patient in one or more of said specific detection positions of said medical institution.

17. The method for managing the medical processes within a medical institution according to claim 3, wherein said step of identifying the path of said patient within said medical institution allows the acquisition of the presence of said patient in one or more of said specific detection positions of said medical institution.

18. The method for managing the medical processes within a medical institution according to claim 4, wherein said step of identifying the path of said patient within said medical institution allows the acquisition of the presence of said patient in one or more of said specific detection positions of said medical institution.

19. The method for managing the medical processes within a medical institution according to claim 5, wherein said step of identifying the path of said patient within said medical institution allows the acquisition of the presence of said patient in one or more of said specific detection positions of said medical institution.

20. The method for managing the medical processes within a medical institution according to claim 6, wherein said step of identifying the path of said patient within said medical institution allows the acquisition of the presence of said patient in one or more of said specific detection positions of said medical institution.
